(19) United States
(12) Patent Application Publication
(10) Pub. No.: US 2020/0375690 A1
(43) Pub. Date: Dec. 3, 2020

(54) BIOMIMETIC DEVICE FOR MANIPULATION OF SOFT TISSUE

(71) Applicant: Bahram Ghaderi, St. Charles, IL (US)

(72) Inventor: Bahram Ghaderi, St. Charles, IL (US)

(21) Appl. No.: 16/888,690

(22) Filed: May 30, 2020

Related U.S. Application Data
(60) Provisional application No. 62/854,809, filed on May 30, 2019.

(51) Int. Cl. A61B 90/40; A61B 17/30

(52) U.S. Cl. CPC A61B 90/40; A61B 2017/00867; A61B 17/30

(57) ABSTRACT

A device for robotic surgery of soft tissue, that comprises a shell for enclosing a surgical area on a patient. The shell has a rim with at least one suction fastener for adhering the shell to the patient. A manipulator arm is positioned within the shell for manipulating the soft tissue in the surgical area, the manipulator arm comprised of at least one flexible module that is capable of omnidirectional bending and elongation.

### CROSS REFERENCE TO RELATED APPLICATIONS

This application claims the benefit of U.S. Provisional Patent Application No. 62/854,809, filed on May 30, 2019, which is incorporated herein by reference in its entirety.

### BACKGROUND OF THE INVENTION

The present invention relates to a medical device for surgery of soft tissue and, in particular, to devices for facilitating manipulation of soft tissues during robotic surgery.

Mastectomy, breast reduction, breast reconstruction and breast enhancement procedures have become commonplace. In typical surgical techniques for breast enhancement, a silicone or saline filled implant device is inserted into the breast after an incision in locations such as the inframammary fold, or periareolar area. In such procedures, it is often necessary for the surgeon to manipulate the soft tissue of the breast and hold it in place to allow easier access to the skin for a clean incision and placement of the breast implant. This minimizes scarring, provides better aesthetic appeal, and prevents postsurgical complications.

It is theoretically possible for robotic devices, such as those sold under the daVinci® trademark (Intuitive Surgical, Inc.—Sunnyvale, Calif.), to execute many breast enhancement procedures. However, it is difficult for a surgeon to operate a robotic surgery apparatus, and also to maintain soft tissues, such as breast tissue, in such a manner to provide the best incision as provided above. It would be desirable to have a device that could, in an automated way, allow manipulation of soft tissues in a surgical procedure.

### SUMMARY OF THE INVENTION

A medical device for surgery of soft tissue in a surgical area on a patient comprises a shell for enclosing the surgical area, a rim formed at a peripheral edge of the shell, and a suction fastener positioned at the rim for adhering the shell to the patient. In an embodiment, a vacuum pump is coupled to the suction fastener. In another embodiment, the suction fastener comprises a chamber with an orifice and a surface surrounding the orifice. In a preferred embodiment, a texture is formed on the surface surrounding the orifice. In an embodiment, the medical device comprises first and second suction fasteners that are independently actuatable to adhere the shell to the patient.

In one embodiment, a medical device for surgery of soft tissue in a surgical area on a patient comprises a shell for enclosing the surgical area, a rim formed at a peripheral edge of the shell, and a manipulator arm within the shell that comprises a suction fastener for adhering the manipulator arm to the soft tissue within the surgical area. In an embodiment, a vacuum pump is coupled to the suction fastener. In another embodiment, the suction fastener comprises a chamber with an orifice and a surface surrounding the orifice. In a preferred embodiment, a texture is formed on the surface surrounding the orifice. In an embodiment, the manipulator arm comprises first and second suction fasteners that are independently actuatable to adhere the manipulator arm to the soft tissue within the surgical area. In an embodiment, the manipulator arm is bendable and/or elongatable. In a preferred embodiment, the bending and/or elongation of the manipulator arm is pneumatically actuatable. In an embodiment, the manipulator arm has variable stiffness. In a preferred embodiment, the variable stiffness is modulated by granular jamming.

In one embodiment, a surgical device for manipulating the soft tissue in a surgical area on a patient comprises a shell for enclosing the surgical area on the patient. A rim is formed at the peripheral edges of the shell, with at least one suction fastener positioned at the rim for adhering the shell to the patient to enclose the surgical area. A manipulator arm is positioned within the shell for manipulating the soft tissue in the surgical area. In one embodiment, the suction fastener can be actuated to adhere the shell to the patient. The suction fastener comprises a chamber with an orifice, the chamber formed of an electroactive polymer. An annular flange surrounds the orifice, the flange having a surface for contacting the patient. The suction fastener is adhered to the patient by actuation of the electroactive polymer. In one embodiment, the shell may comprise multiple suction fasteners that are divided into first and second sets of suction fasteners that are independently actuated.

In one embodiment, a surgical device for manipulating the soft tissue in a surgical area on a patient comprises a manipulator arm that comprises at least one flexible module having an elastomeric central channel and at least one pneumatic chamber adjacent the central channel, where the actuation of the pneumatic chamber bends the flexible module. In an embodiment, a suction fastener is positioned on the manipulator arm for adhering the manipulator arm to the soft tissue within the surgical area.

### BRIEF DESCRIPTION OF THE DRAWINGS

FIG. 1 is a front elevation view of a vacuum shell apparatus, showing how it would be mounted in a breast augmentation or other surgical procedure.

FIG. 2 is a side section, detail view of a rim portion of the vacuum shell apparatus of FIG. 1.

FIG. 3 is a top view of an alternative embodiment of a vacuum shell apparatus.

FIG. 4 is a side section, detail view of the embodiment of a vacuum shell apparatus of FIG. 3, showing the vacuum hose assembly.

FIG. 5A is a front elevation view of yet another embodiment of a vacuum shell apparatus having moveable negative pressure points, showing how it would be mounted in a breast augmentation or other surgical procedure.

FIG. 5B is a side section view of the embodiment of a vacuum shell apparatus of FIG. 5A.

FIG. 6 is a side section, detail view of the embodiment of a vacuum shell apparatus of FIG. 5B, showing a moveable negative pressure point positioned over a breast.

FIG. 7A is a top perspective view of the embodiment of the vacuum shell apparatus of FIG. 5A.

FIG. 7B is a bottom perspective view of the embodiment of a vacuum shell apparatus of FIG. 7A.

FIG. 8 is a top perspective view of the embodiment of the vacuum shell apparatus of FIG. 7A, showing how it would be mounted in a breast augmentation or other surgical procedure.

FIG. 9 is a bottom perspective view of yet another embodiment of a vacuum shell.

FIG. 10 is a bottom perspective view of yet another embodiment of a vacuum shell, comprising biomimetic manipulators.

### DETAILED DESCRIPTION OF THE INVENTION

One embodiment of the soft tissue enlargement apparatus 10 may be generally comprised of an open shell 12 having a rim 14 and a vacuum port 15 which leads through vacuum hose 15a to a vacuum pump 16 for creating a vacuum within the shell 12. Although the vacuum pump assembly 16 may be a separate hand-held pump in one variant embodiment, in the preferred embodiment the vacuum pump assembly 16 is a self-contained vacuum pump with an independent power source, pressure sensor, and servomechanism for driving, regulating and controlling the vacuum pump 16.

The shell 12 may preferably be comprised of a rigid clear plastic material such as polycarbonate, which is sufficient to withstand vacuum pressure within the shell 12. Preferably, the shell 12 would be designed to fit over the chest area of a patient, and can be any shape, including a dome, to effect this purpose. Other specific embodiments of the shell may be configured to fit over a human breast, or as a bra shape.

The shell 12 may be designed to leave 1-5 inches of space between the area of operation and the shell, to provide space for tissue to expand, and sufficient space for surgical instruments to operate effectively. However, the shell 12 should not be so large so as to require a large pump to maintain sufficient negative pressure under the shell 12.

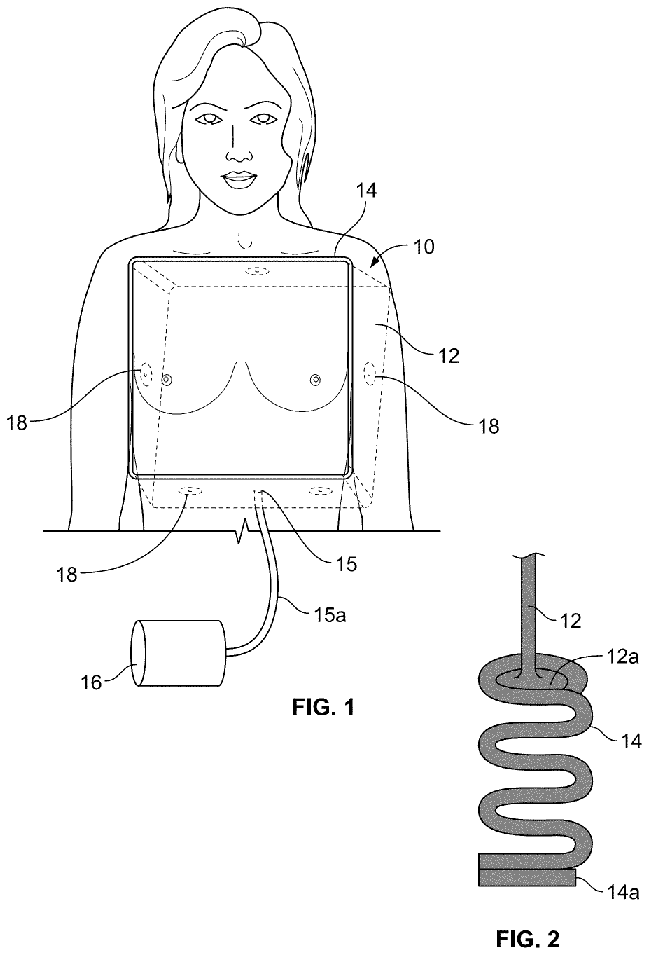

As shown in detail in FIGS. 1 and 2, the rim 14 may be a gasket disposed around the edges 12a of the shell 12, and may be comprised of a flexible, preferably soft material, such as rubber capable of forming a seal when contacted with skin. In a preferred embodiment, this rim 14 may be a silicone gel cushion or other soft, conforming type material. Petroleum jelly may also be used to supplement or supplant the rim. The rim 14 may be coated with a pressure sensitive adhesive material 14a, such as a double-sided adhesive with a peelable contact paper, to assist in maintaining sealing contact with the skin. The rim 14 may be sized to extend an inch or more from the edges 12a of the shell 12 in order to adapt to differing body profiles.

Regulation of the negative pressure within the shell 12 is essential to prevent contusions caused by rupturing capillaries adjacent the surface of the skin. Medical data suggest that these contusions will not occur if negative pressure within the shell is maintained at less than 20 mmHg. Thus, the vacuum pump 16 may be regulated to control the vacuum within the shell to within this limit. In addition, skin ulceration may occur if excessive contact pressures are applied thereto. Medical data suggest that a negative pressure less than 20 mmHg may be applied indefinitely without such ulceration. However, contusions may occur due to positive contact pressures upon the skin at pressures above this ulceration limit.

The shell 12 may be provided with one or more sealing instrument ports 18 that are seals that can be reclosably penetrated by an instrument, such as an instrument driven by a robotic surgery device, such as a DaVinci surgical robot device. Such ports 18 may be formed by a circular disk of a flexible material such as a gel, having a pinhole at the center. The port 18 may be sealed against the environment, but may be expanded to fit around a surgical instrument when the pinhole is penetrated thereby. Other embodiments of sealed instrument ports may also be used.

In operation, the shell 12 may be placed over the surgical area, such as a chest area, such that the rim 14 is provides a seal with the patient's skin. The vacuum pump 16 may be used to provide negative pressure through port 15 and hose 15a. The negative pressure throughout the shell will allow the soft tissue of the breast to expand, and expose more of the skin to allow a clean incision and manipulation during surgery.

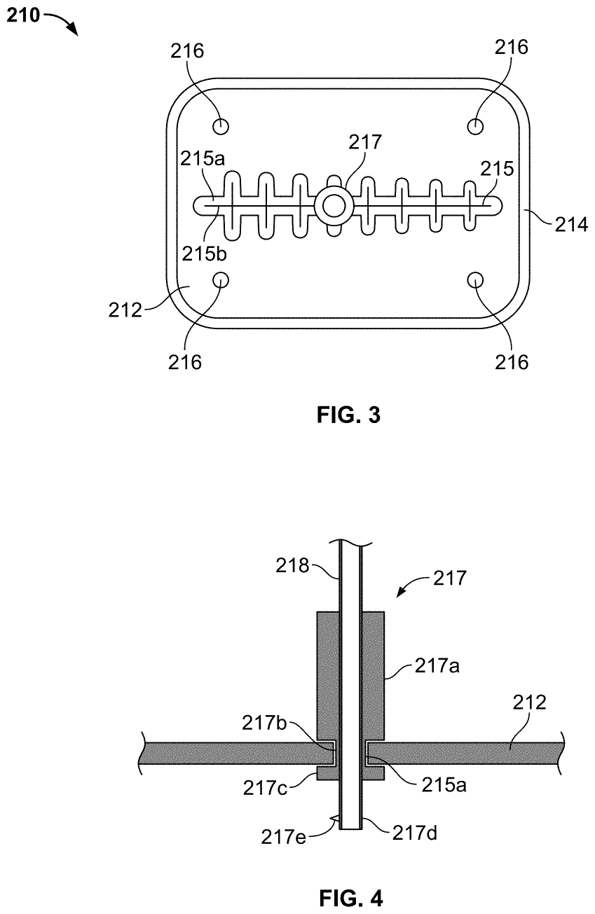

While the first embodiment allows for a constant negative pressure throughout the volume of the shell 12, it would be useful to modify the device so as to allow localized negative pressure, from the vacuum hose, at different points during surgery. For example, in a breast augmentation operation, the surgeon may want to provide concentrated negative pressure on the breast being operated on. A second specific embodiment is shown in FIGS. 3 and 4 showing one way to allow the surgeon to both provide negative pressure to the entire shell 12, and provide a concentration of negative pressure in a localized area by adjusting the placement of the vacuum port within the shell.

As seen in FIG. 3, a top view, a shell 212, similar to shell 12 in FIG. 1, may be designed to fit over the surgical area, in particular, the chest area for a breast enlargement procedure. The shell 212 may have a rim 214 essentially similar to the rim 14 described in FIG. 2, and one or more instrument ports 216 similar to instrument ports 18 disclosed in FIG. 1. The vacuum port 215, rather than being a single hole 15 as shown in FIG. 1, may be a slot or network of slots 215a in the shell that can allow a vacuum hose assembly 217 to travel at varying points across the shell 212. In the slots 215a of vacuum port 215, may be disposed a flexible sealing material, such as a gel, with a pin slit 215b extending lengthwise along all the slots forming the vacuum port 215. The pin slits, when not pierced by hose assembly 217, may be sealed by the expansion of the sealing material to allow negative pressure formation inside shell 212. The vacuum hose assembly 217 can be moved anywhere in the slots of vacuum port 215 to apply vacuum in a localized area.

A schematic drawing of a vacuum hose assembly 217 is shown at FIG. 4. As shown in FIG. 4, hose assembly 217 comprises cylindrical sleeve 217a having a diameter slightly greater than the slots forming vacuum port 215. The sleeve 217a may have a narrow portion 217b which is the portion that reclosably pierces the sealed slots 215a. The narrow portion 217b has a length approximately equal to the thickness of the shell 212. The sleeve may further comprise a wide end flange 217b that can be detachably connected to the narrow portion 217b, so that the sleeve 217a can be securely mounted to the slots 215a of the vacuum port 215, with the narrow portion 217b piercing the slits 215b. Inside the sleeve a vacuum hose 217 may be sealingly mounted, such that the vacuum hose can travel in the assembly 217 along the slots 215a around the shell 212. As the assembly 217 moves through slots 215a, the portion of the slits 215b that were pierced by narrow portion 217b may reclose as the hose assembly 217 moves to another location. In addition, the hose 217d may be slid through the sleeve 217a to allow the hose to be moved nearer or further to contact with the tissue being operated upon. The hose 217d may have a tab 217e at the end to prevent the hose from being drawn out of the sleeve during operation.

In this manner, the surgeon operating a robotic surgery device can place the shell 212 against the patient, apply negative pressure throughout the shell 212, and provide localized negative pressure to specific areas under the shell 212, to help locally expose the skin and hold soft tissue in place, in the exact location where the surgeon is operating, without requiring extensive manual manipulation of the soft tissue. All this may be done while maintaining negative pressure throughout the shell 212 via the vacuum pump 216.

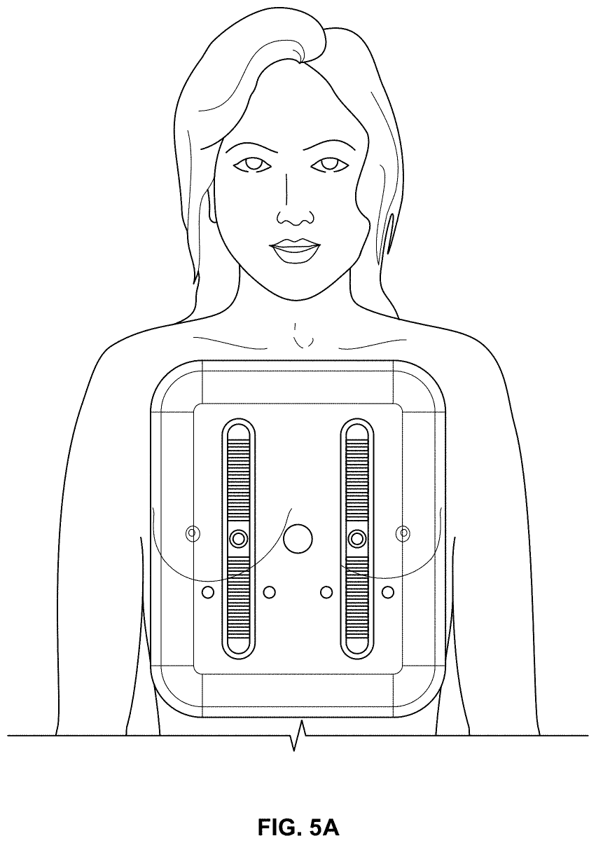

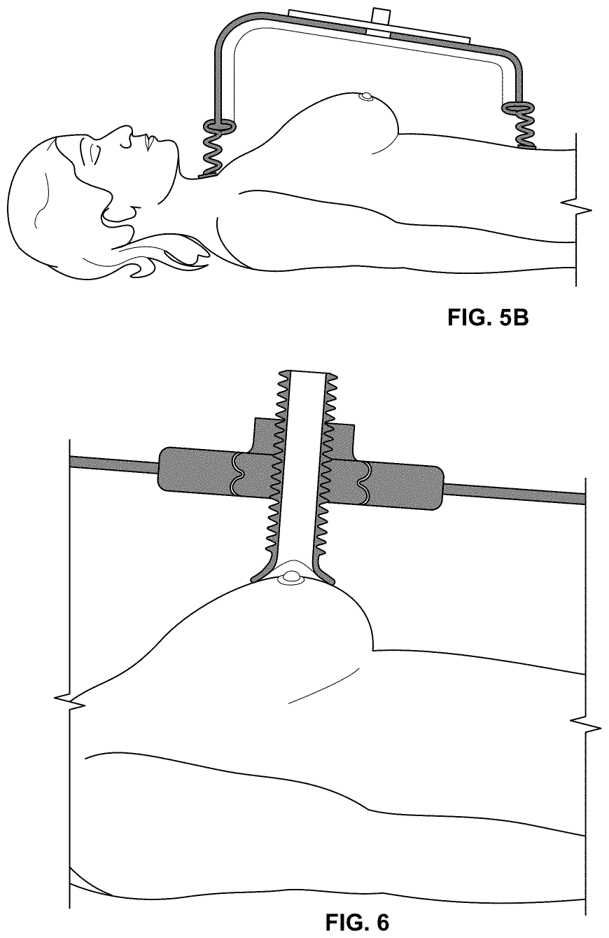

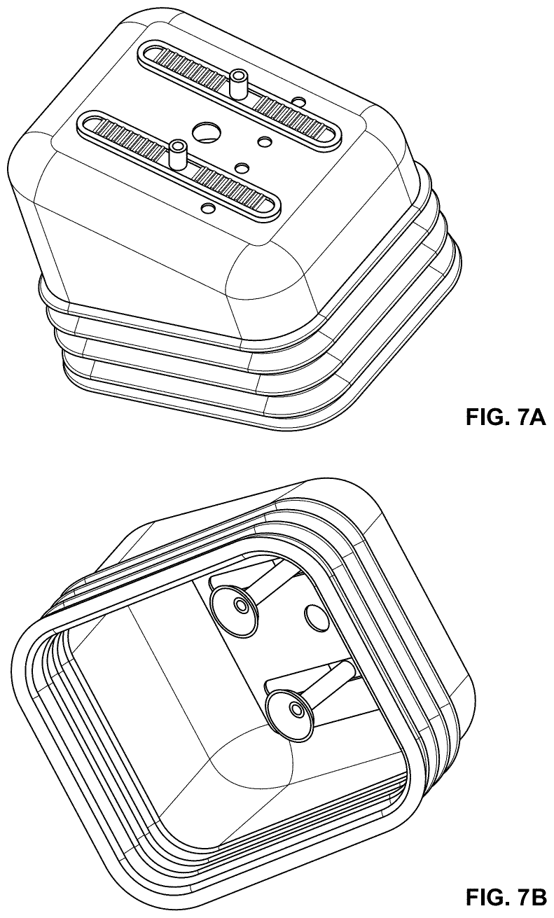

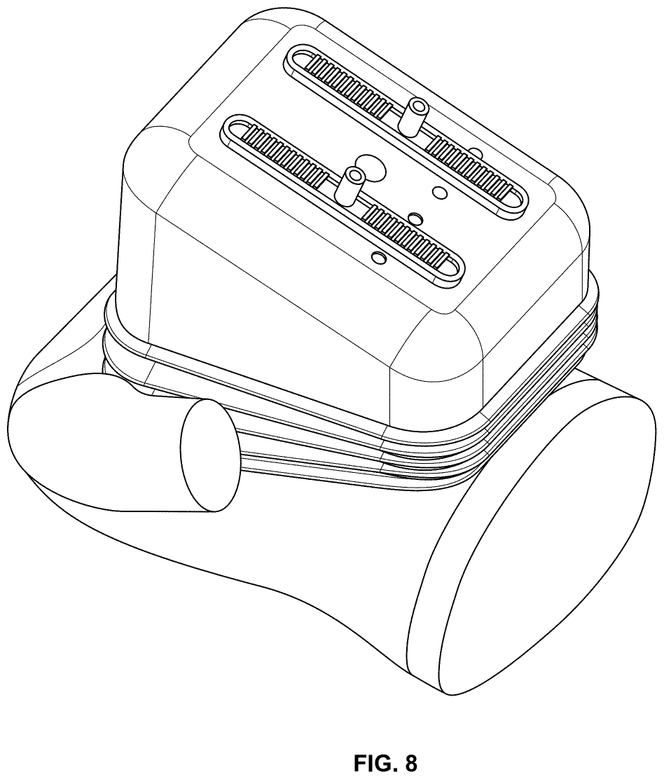

Referring to FIGS. 5A, 5B, 6, 7A, 7B and 8, an alternative embodiment of vacuum shell apparatus is shown, that provides one or more moveable negative pressure points so that the breast or other soft tissue can be maneuvered to allow for surgeries such as mastectomy and reconstruction to be performed with more accuracy and less scarring.

When the shell is secured to the patient by the vacuum within the volume of the shell, the operator must exercise some care in regulating the negative pressure to avoid causing injury to soft tissues, such as ruptured capillaries and ulceration as previously discussed. The shell may also be sealed to the patient using adhesives. However, many adhesives have reduced adhesion in the presence of fluids, such as antiseptics, blood, and saline or other irrigation fluids that are typical of surgical procedures. Adhesives may also lose adhesion after they are removed, which limits the ability to reposition the shell during surgery. Furthermore, exposure to adhesives may cause skin irritation, such as contact dermatitis or allergic dermatitis. Thus, it would be desirable to provide alternative methods of securing the shell to the patient, that can function in the presence of fluids and that can be removed and repositioned without losing adhesion.

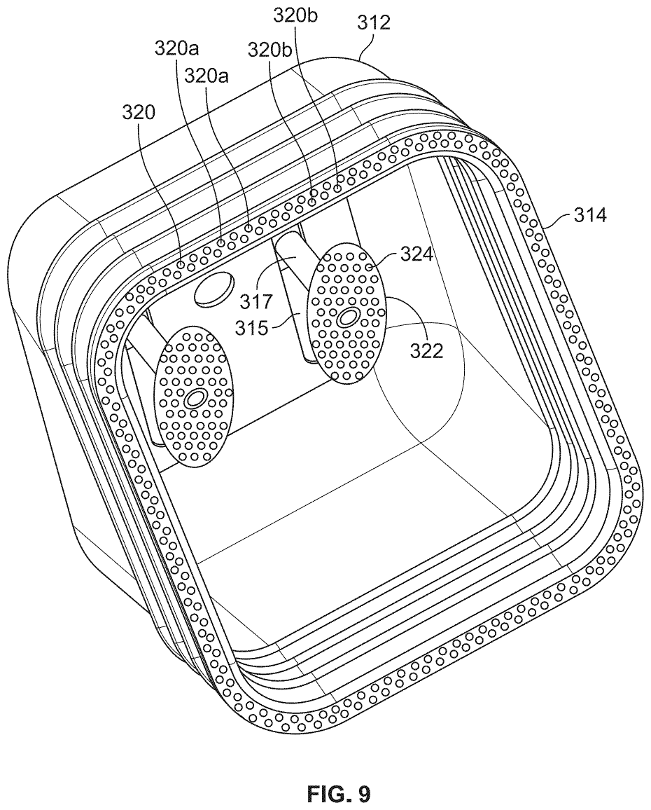

Referring to FIG. 9, a shell 312 is shown that incorporates alternative systems for adhering the shell to the skin of a patient and for manipulating soft tissue. In one embodiment, shell 312 may be adhered to the skin by a series of suction fasteners 320 positioned at multiple points around rim 314. Suction fasteners 320 may comprise openings coupled to a vacuum pump. Alternatively, suction fasteners 320 may be conventional suction cups formed of an elastomeric material. In a preferred embodiment, suction fasteners 320 are biomimetic and share one or more features of octopus suckers which have a number of advantages, such as the ability to operate in water environments and to adhere to irregular shapes. For example, suction fasteners 320 may have pattern or other texture formed in the contact surface, such as radial grooves to improve adhesion under wet conditions, concentric grooves to improve sealing contact with the patient's skin. In one embodiment, the contact surface of suction fastener 320 has a texture formed of micro peg-shaped features or denticles that create a network of channels for distribution of the vacuum forces to the edges of the contact surface. The denticles also create a rough surface to enhance friction and resist shear forces that may cause lateral movement of suction fastener 320 and shell 312.

In another embodiment, suction device 320 may have a similar structure to an octopus sucker—e.g., a chamber with an orifice surrounded by an annular flange that forms the contact surface of the sucker. A protuberance is formed in the interior ceiling of the chamber. When suction device 320 is engaged with a substrate such as the patient's skin, the protuberance seals the orifice and isolates the space between the contact surface and substrate from the space within the chamber. Sealing the orifice allows the chamber to relax while negative pressure is maintained between the contact surface and substrate. Adhesive materials have been developed that comprise pads of polyurethane-based elastomers that are molded with an array of biomimetic cylindrical microsuckers having a chamber roof that is designed to come into contact with the substrate surface when the microsuckers are engaged. The adhesive pads are found to adhere to hairy skin in wet and dry conditions, with high repeatability through multiple cycles of adhesion and removal.

The seal between the protuberance and orifice may be improved by forming suction fastener 320 with materials having different elasticities. For example, the protuberance may have reduced elasticity compared to the annular flange and orifice. Ridges or similar structures may be formed on the surfaces of the protuberance and orifice to improve the seal. Other biomimetic features may be incorporated in suction fastener 320. For example, squid suckers comprise a piston-like structure inside a rigid central body. Forces that pull the sucker away from the surface tend to raise the piston away from the surface which increases negative pressure within the sucker.

In yet another embodiment, suction fastener 320 may be actuated to adhere to a substrate surface. For example, where suction fastener 320 is an opening coupled to a vacuum pump, the suction fastener may be actuated by coupling/uncoupling the opening to the vacuum, or turning on/off the pump. Alternatively, suction fastener 320 may be a biomimetic sucker formed using an electroactive polymer that changes in size or shape when an electric field is applied. Suction fasteners have been developed that comprise a chamber formed of multiple layers of a dielectric elastomer. When voltage is applied, the electrodes are attracted to each other, which reduces the thickness of the chamber wall and increases in the enclosed volume of the chamber. If the suction fastener is sealed to a substrate surface, the pressure within the chamber is reduced and an adhesion force is generated. Similar thermally actuated adhesive materials have also been developed. Adhesive pads of polydimethylsiloxane are formed with an array of microscale suckers that comprise pores or pits coated with a thermally responsive polymer. At room temperature, the walls of the pores are in a relaxed open state. When heated, the walls of the pores contract, creating suction. Thermoelectrically actuated shape memory alloys may also provide a means for actuating a change in the size or shape of a sucker chamber.

FIG. 9 shows an array of suction fasteners 320 positioned around rim 314 of shell 312. Suction fasteners 320 are preferably arranged in pattern that permits different sets of suction fasteners to be independently engaged to secure shell 312 to the patient. For example, the array of suction fasteners 320 may comprise an outer row of suction fasteners 320a and an inner row of suction fasteners 320b that are independently actuatable. Alternately actuating the outer or inner rows of suction fasteners 320a, 320b to adhere shell 312 to the patient reduces the length of time that a particular area is exposed to vacuum or suction attachment and reduces the risk of injury to the skin or soft tissues. In addition, the use of a large number of smaller suction fasteners may also increase their ability to adhere to the substrate surface, as the pressure differential is believed to be inversely correlated with sucker size.

Biomimetic suction fasteners may also be used for manipulation of tissues within the shell. For example, vacuum hose 217d may be replaced by a manipulator arm 317 positioned within a port 315, that has one or more flanges 322. The surface of flange 322 may be provided with one or more suction fasteners 324 as previously described. In operation, arm 317 is positioned over and lowered into contact with the tissue to be manipulated. Suction fasteners 324 come into contact with and adhere to the tissue, and arm 317 may be withdrawn or otherwise moved within the port 315 to manipulate the tissue. Flange 322 may also have an array of suction fasteners 324 which are arranged in a pattern that allows alternating sets of suction fasteners that are independently actuatable to engage the tissue as previously described.

As shown in FIG. 9, manipulator arm 317 is a rigid rod that is generally constrained to movement in straight lines, within the slot or network of slots of port 315. The limitations on the maneuverability of arm 317 may require the use of multiple instruments to adequately access and manipulate the tissues for some procedures. Thus, it would be desirable to provide shell 312 with a manipulator arm that has greater freedom of movement.

In one embodiment, the manipulator arm comprises a biomimetic manipulator that resembles an octopus tentacle—i.e. that is capable of bending in any direction, elongating, and adapting to different geometries. An example of a biomimetic manipulator is the STIFF-FLOP manipulator developed by The Biorobotics Institute of Sant′Anna School of Advanced Studies, as described in M. Cianchetti, et al., STIFF-FLOP Surgical Manipulator: Mechanical Design And Experimental Characterizaton Of The Single Module, 2013 IEEE/RSJ Int'l Conf. on Intell. Robots and Sys. (IROS), Nov. 3-7, 2013 (Tokyo, Japan), which is incorporated herein by reference in its entirety.

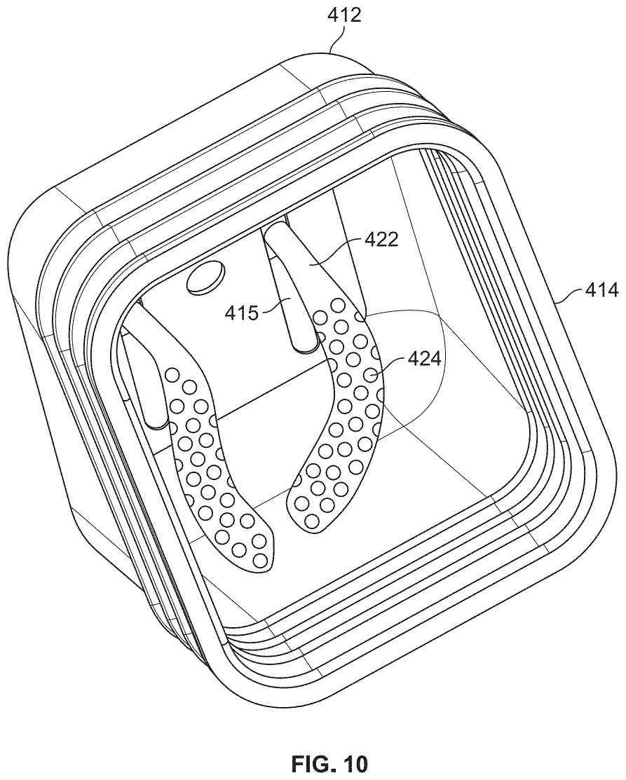

FIG. 10 shows an embodiment of a shell 412 with one or more biomimetic manipulator arms 422 that are capable of omnidirectional bending and/or elongation. For example, manipulator arm 422 may be comprised of one or more flexible modules that are pneumatically actuatable. Each module has an elastomeric central channel, at least one pneumatic chamber positioned adjacent to the central channel, and a sheath of elastomeric material enclosing the central channel and pneumatic chamber. The sheath may have a bellows shape to allow the module to be extended and retracted. When the pneumatic chamber is actuated and expands, the adjacent portion of the central channel is elongated, which causes the central channel to bend. In a preferred embodiment, the central channel is surrounded by three pneumatic chambers in a uniform radial arrangement. The module may be bent in any direction and/or elongated by actuation of the pneumatic chambers singly, in pairs, or all at once. The module may be configured to limit lateral (radial) expansion of the pneumatic chambers, such as a braided sheath that maximizes longitudinal expansion and promotes bending and/or elongation. The flexibility and maneuverability of the manipulator arm 422 reduces the need for additional instrument ports to provide access to the surgical area, thereby increasing the reliability of shell 412.

In another embodiment, the stiffness of manipulator arm 422 may be modulated. Manipulator arm 422 is initially flexible for movement and positioning of the arm. Once manipulator arm 422 is properly positioned and suction fasteners 324 are adhered to the tissues, the arm becomes rigid to support to support and manipulate the tissues. For example, the stiffness of the flexible module may be modulated by filling the central channel with particles that have the property of granular jamming—i.e. where an aggregate of particles increases in viscosity with increasing aggregate density. Under normal conditions the aggregate is relatively fluid, which permits the central channel and module to bend freely. Jamming is induced by applying a vacuum to the central channel, which increases the particle density of the aggregate. The resulting crowding of particles makes the aggregate behave as a solid and increases the rigidity of the central channel and module.

In a preferred embodiment, the surface of manipulator arm 422 is provided with one or more suction fasteners 424 for engaging the tissues in the surgical area. Suction fasteners 424 may be disposed on one side or segment of the surface of manipulator arm 422, or may be disposed over substantially the entire surface to take advantage of the omnidirectional movement of the manipulator arm. In one embodiment, suction fasteners 424 are arranged in a pattern or array that includes alternating sets of suction fasteners to be independently actuated to engage the tissue, as previously described.

Manipulator arm 422 may be combined with a shell 412 that is secured to the patient by a variety of different means. Rim 414 may be configured similarly to rim 14—e.g., with a flexible or conformable gasket and/or with an adhesive material. Rim 414 may also be configured with a series of suction fasteners, as described for rim 314.

There are various changes and modifications which may be made to the invention as would be apparent to those skilled in the art without departing from the concept and scope of the invention. While the invention has been described in terms of the preferred embodiment of breast surgery, those of skill in the art will readily appreciate that the apparatus and methods may be adapted to other soft tissue surgery, and that modifications may be made as to the means for adjusting the location of a vacuum within the shell. These changes or modifications are included in the teaching of the disclosure and it is intended that the invention be limited only by the scope of the claims appended hereto.

What is claimed is:

1. A medical device for surgery of soft tissue in a surgical area on a patient, comprising:
a shell for enclosing the surgical area;
a rim formed at a peripheral edge of the shell; and
a suction fastener positioned at the rim for adhering the shell to the patient.

2. The medical device of claim 1, further comprising a vacuum pump coupled to the suction fastener.

3. The medical device of claim 1, wherein the suction fastener comprises an electroactive polymer that changes the shape of the suction fastener in response to an electric field.

4. The medical device of claim 1, wherein the suction fastener is thermally actuated to change shape.

5. The medical device of claim 4, wherein the suction fastener comprises a thermoelectrically actuated shape memory alloy.

6. The medical device of claim 1, wherein the suction fastener comprises a chamber with an orifice and a surface surrounding the orifice.

7. The medical device of claim 6, wherein a texture is formed on the surface surrounding the orifice.

8. The medical device of claim 7, wherein the orifice is surrounded by an annular surface and the texture comprises at least one radial groove.

9. The medical device of claim 7, wherein the orifice is surrounded by an annular surface and the texture comprises at least one concentric groove.

10. The medical device of claim 7, wherein the texture comprises a channel formed of denticles.

11. The medical device of claim 1, comprising first and second suction fasteners that are independently actuatable to adhere the shell to the patient.

12. A medical device for surgery of soft tissue in a surgical area on a patient, comprising:
a shell for enclosing the surgical area;
a rim formed at a peripheral edge of the shell; and
a manipulator arm within the shell, comprising a suction fastener for adhering the manipulator arm to the soft tissue within the surgical area.

13. The medical device of claim 12, further comprising a vacuum pump coupled to the suction fastener.

14. The medical device of claim 12, wherein the suction fastener comprises a chamber with an orifice and a surface surrounding the orifice.

15. The medical device of claim 14, wherein a texture is formed on the surface surrounding the orifice.

16. The medical device of claim 12, wherein the manipulator arm comprises first and second suction fasteners that are independently actuatable to adhere the manipulator arm to the soft tissue within the surgical area.

17. The medical device of claim 12, wherein the manipulator arm is bendable and/or elongatable.

18. The medical device of claim 17, wherein the bending and/or elongation of the manipulator arm is pneumatically actuatable.

19. The medical device of claim 17, wherein the manipulator arm has variable stiffness.

20. The medical device of claim 19, wherein the variable stiffness is modulated by granular jamming.
